(19) United States
(12) Patent Application Publication
(10) Pub. No.: US 2024/0000995 A1
(43) Pub. Date: Jan. 4, 2024

(54) RAPIDLY RESORBABLE INTRAVASCULAR IMPLANT

(71) Applicant: BIOTRONIK AG, Buelach (CH)

(72) Inventors: Heinz Mueller, Rostock (DE); Marion Philine Zumstein, Horgen (CH)

(21) Appl. No.: 18/254,312

(22) PCT Filed: Jan. 11, 2022
(86) PCT No.: PCT/EP2022/050465

§ 371 Date: May 24, 2023

(30) Foreign Application Priority Data
Jan. 29, 2021 (DE) 202021100450.9

(51) Int. Cl. A61L 27/04; A61L 27/38

(52) U.S. Cl. CPC A61L 27/047; A61L 27/3839

(57) ABSTRACT

A vascular support implant is formed from a magnesium alloy. The alloy includes Zn and/or one of the following elements: Ca, Sr. The Zn content is ≤1.5 wt. % and a Ca content is ≥0.25 wt. %.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2022/050465, which was filed Jan. 11, 2022, which application claimed priority from German Application Serial Number 202021100450.9, which was filed Jan. 29, 2021.

### FIELD OF THE INVENTION

The invention relates to an implant in the form of a vascular support. Such a vascular support is also referred to as a scaffold and is generally designed as a circumferential structure made of struts that are connected to one another and form the cells of the scaffold.

### BACKGROUND

Rapidly biodegradable vascular supports for sealing vulnerable plaques or biodegradable vascular supports as temporary flow diverters for treating aneurysms are not available in the market.

If permanent or slowly degradable vascular supports are used for this purpose, even though an implant is only required for a very short period of time, the implant (for example, due to potential thromboses) or necessary long-term medication (for example, additional hemorrhage due to the administration of thrombocyte aggregation inhibitors) poses a potential longer-term or permanent risk in the least favorable case, for example in the case of intracranial aneurysms with permanent implants.

The biodegradable vascular supports/scaffolds available in the market were primarily developed to preserve as great a radial force as possible for the longest possible time, which is not even required for the above-described applications.

The disadvantages of these implants, which are predominantly made of plastic (PLLA), are, in principle, high wall thicknesses, in many instances poor adaptation to the shape of the vessel (malapposition), very long degradation times and, resulting from all these factors, a relative high risk of thrombosis.

Mg-based vascular supports additionally result in a permanent degradation product; however, the tendency to cause thrombosis appears to be drastically reduced compared to permanent metals and plastic materials, regardless of the wall thickness.

### SUMMARY OF THE INVENTION

A vascular support implant is formed from a magnesium alloy. The alloy includes Zn and/or one of the following elements: Ca, Sr. The Zn content is ≤1.5 wt. % and a Ca content is ≥0.25 wt. %.

### DETAILED DESCRIPTION OF THE PREFERRED EMBODIMENTS

The invention provides an implant or a vascular support that possess rapid and substantially residue-free degradation. In particular, a rapidly biodegradable vascular support is provided, which has mechanical integrity that is limited in terms of time and which can be degraded after the mechanical integrity has been lost, and preferably is used to seal vulnerable plaques or as a temporary flow diverter for treating aneurysms.

A preferred vascular support, in particular for implantation into a vulnerable plaque or for disrupting the flow into an aneurysm, is fully biodegraded within a time period of less than 360 days, in particular of less than 180 days, preferably of less than 90 days. The implant includes a magnesium alloy or is made thereof, including Zn (zinc) and/or one of the following elements: Ca (calcium), Sr (strontium).

The invention thus in particular provides an implant or a scaffold/vascular support that can be implanted into an existing vulnerable plaque, so that uncontrolled rupturing of the plaques, and a potentially subsequent thrombotic event, can be prevented, and the implant can, thereafter, be completely biodegraded within a time period of less than 360 days, preferably of less than 180 days, and ideally in less than 90 days.

Furthermore, one embodiment of the invention relates to an implant or a vascular support/scaffold that, when an aneurysm exists, disrupts the flow into the arising cavity which maintains this aneurysm from a fluid point of view, so that a flow-free zone can arise in the space of the former aneurysm and the cavity can be incrementally closed by coagulation and cell colonization. In the process, depending on the requirements of the therapy, the implant is to be completely dissolved after a time period of less than 360 days, less than 180 days, or even less than 90 days, so that the risk of thrombosis caused by a foreign object or a degradation product is avoided, especially in intracranial vessels. The invention relates in particular to an implant for treating local vascular diseases that require only short-term mechanical stabilization (for example vulnerable plaques or aneurysms).

According to one embodiment of the invention, it is provided that the implant is made of or includes a biodegradable magnesium alloy, in which the time until full biodegradation can be varied by way of the content of the involved alloying elements. The implant (for example is vascular support) can be coated with a polymer, and this polymer can contain a pharmaceutical drug.

As a result of rapid degradation of an implant in the two applications mentioned above, the time during which a patient has to be treated with thrombocyte aggregation inhibitors (dual antiplatelet therapy, DAPT) is also decreased. A shorter therapy duration reduces the risk of undesirable hemorrhage. In addition, minimal and only brief mechanical loading of the vessel decreases a provocation of the excess proliferation of neointima and potential stenosing of the affected vascular section associated therewith.

According to one embodiment of the invention, a biodegradable vascular support is provided, which can be implanted into the vascular wall at the appropriate location by way of a balloon. Such a vascular support is preferably made of a biodegradable metal. To achieve complete dissolution after less than 360 days, a magnesium alloy (Mg alloy for short) is preferably used for this purpose. Suitable mg alloys are preferably those alloys that contain Ca and/or Zn.

Surprisingly, it was additionally shown in animal experiments that this group of alloys can be degraded to a very large degree after a comparatively shorter implantation duration, and can be fully degraded in the case of the more rapidly degrading alloys that have higher Zn contents, without leaving behind a degradation product or a relevant amount of residues.

This finding was not to be expected insofar as other previously examined alloys, specifically magnesium alloys, that contain rare earths or aluminum as the alloying elements have a longer degradation time, and leave a degradation product behind.

The degradation kinetics can be set by way of the Zn content or by way of the Zn/Ca ratio, in general the following applying:
the higher the Zn content, the more rapidly the degradation progresses;
the lower the Zn content, the more slowly the degradation progresses;
the higher the Zn/Ca ratio, the more rapidly the degradation progresses;
the lower the Zn/Ca ratio. the more slowly the degradation progresses.

According to one embodiment of the invention, it is provided that the Mg alloy is a Mg alloy having a Zn content between 1.5 wt. % and 20.0 wt. % Zn and including 0 wt. % to 1 wt. % Ca.

According to a further embodiment, preferably Mg alloys including between 1.5 wt. % and 6.0 wt. % Zn are to be selected, in particular for full thermomechanical processability, since all the Zn can be brought into solution at up to 6.0 wt. % Zn.

According to a further embodiment, Mg alloys having a Zn content of ≥1.5 wt. % and a Ca content of <0.5 wt. % are preferred, in particular for rapid and substantially reliable full dissolution.

According to a further embodiment of the invention, it is provided that Mg alloys including more than 6.0 wt. % Zn are to be selected, in particular for the high strength possible, since here a permanently precipitated intermetallic phase of Mg and Zn is present, and very strong particle hardening is also achieved at room temperature due to the high percent by volume.

The above-described Mg alloys are also referred to as high Zn alloys hereafter. These alloys are in particular suitable for achieving as complete a degradation as possible in less than 360 days, possible also in considerably less time, as was able to be demonstrated in animal experiments.

According to further embodiments of the invention, it is provided that the Mg alloy is one of the following Mg alloys:
A Mg alloy having a Zn content of ≤1.5 wt. % and a Ca content of preferably ≥0.25 wt. %.
A Mg alloy having a Zn content of 0 wt. % to 0.1 wt. % and a Ca content of preferably 0.1% wt. % to 1.0 wt. %.
A Mg alloy having a Zn content of 0.1 wt. % to 0.75 wt. % and a Ca content of preferably 0.1 wt. % to 0.75 wt. %.

In contrast to the high Zn alloys, these Mg alloys are also referred to hereafter as low Zn alloys. It was demonstrated based on animal experiments in this regard that such low Zn alloys can be used to achieve substantial degradation of the implant between 90 days and a time period of approximately 1 year, as well as the longest possible structural stability, that is, the slowest possible degradation.

Furthermore, according to one embodiment of the invention, it is provided that the calcium alloy type is replaced with strontium in the respective high Zn alloy or the respective low Zn alloy.

If it is desired that an implant according to the invention is to be stable initially for a certain period of time, but is to degrade as rapidly as possible thereafter, it is provided, according to a further embodiment, to additionally coat the respective high Zn alloy or the implant made thereof with a biodegradable polymer. In this case, for example, an implant in the form of a vascular support is initially protected by the polymer against corrosion. Following degradation of the polymer, which has progressed far enough for the physiological medium to penetrate the layer, the vascular support or the implant degrades, which takes place particularly rapidly in the case of the high Zn alloys. Suitable biodegradable polymers are listed below.

If it is desired to optimize the mechanical properties of one of the aforementioned metal alloys (for example by way of grain refinement) or to further expedite the degradation kinetics thereof, for example for reasons related to the mechanical properties, when the composition being is fixedly defined, this can be achieved, according to a further embodiment of the invention, by adding one or more micro-alloying elements.

According to one embodiment, such micro-alloying elements are typically used in contents of less than 1 wt. %, preferably in contents of 0.01 wt. % to 0.1 wt. %.

This can preferably involve the addition of one or more of the elements Ag, Fe, Mn, Si, for example, wherein in particular Mn and Si, preferably in combination, can be used to generate specific, strength-enhancing and corrosion-expediting intermetallic phases.

This definition of limits for the contents of possible micro-alloying elements applies to both alloy types.

To obtain particularly advantageous mechanical properties, one embodiment of the implant according to the invention is provided, which has a fine-grained microstructure, having a grain size of no more than 7.5 μm, preferably <5 μm, and in particular preferably <2.5 μm.

During the dilatation of plaques, particles (for example components of the soft, fat-like plaques or also small, fully or partially calcified particles) can be released. To additionally offer particularly high protection against such a possible release of particles, the implant or the vascular support can additionally be provided with a net. This net or covering can be attached to the inside or outside.

According to one embodiment, the net is made of one of the aforementioned biodegradable metal alloys, wherein a preferred diameter of the wires that are used ranges between 10 μm and 100 μm, and/or wherein the preferred size of the mesh (distance between the wires) ranges between 10 μm and 1 mm.

According to one embodiment, the net is made of one of the aforementioned biodegradable metal alloys, wherein a preferred diameter of the wires that are used ranges between 10 μm and 100 μm, and/or wherein the preferred size of the mesh (distance between the wires) ranges between 10 μm and 1 mm, wherein the net is additionally coated with a biodegradable polymer.

At least one of the following polymers can be used as the biodegradable polymer, which can be used as a coating on the resorbable base body of the implant, in particular of the vascular support, within the above-described meaning: polylactide, polyglycolide, polyanhydride, polyhydroxybutyrate, polycaprolactone, polydioxanone, a poly(trimethylene carbonate)-based polymer, polyphosphazene, polyhydroxyalkonates, polyanhydride, polyacetal, polycarbonate, poly(ether ester); a copolymer of the aforementioned polymers, a mixture of the aforementioned polymers, or a blend of the aforementioned polymers.

The following are considered to be particularly suitable: poly(L-lactide), poly)D,L-lactide), poly(D,L-lactide-co-glycolide), polyhydroxybutyrate, polycaprolactone, copolymers of the aforementioned polymers.

According to one embodiment of the invention, the layer thickness of the polymer layers can be 50 nm to 25 μm, layer thicknesses of 200 nm to 10 μm being preferred, and those of 1 μm to 5 μm being particularly preferred.

To be able to achieve as effective covering of a vulnerable plaque or of an aneurysm as possible by way of the base body of the vascular support, additional designs can optionally be preferred for the vascular supports which have an increased so-called “metal to artery ratio” compared to conventional vascular supports. The metal to artery ratio describes the percentage of the vessel wall that is covered over the length across which the implant extends in the vessel. Suitable metal to artery ratios for implants or vascular supports according to the invention are in a range of above 15%, preferably above 25%, and in cases in which the greatest possible coverage is desired, up to 50%. (The information always relates to the coverage in percent after implantation).

Further examples of the invention using high Zn alloys
Vascular support made of a MgZn5Ca0.25 alloy for dilating a vulnerable plaque in a coronary vessel, of which ≥95% of the volume of the body is degraded residue-free within approximately 90 days; the vessel support has a metal to artery ratio of 25% and a 3 μm thick layer made of polylactide.
Vascular support made of a MgZn2.5Ca0.5 alloy, which is used as a flow diverter for an intracranial aneurysm, can maintain the geometric integrity thereof for ≥2 weeks, and of which ≥80% of the volume of the body is degraded residue-free within approximately 180 days, wherein the vascular support is furthermore coated with a layer made of a polylactide/polycaprolactone 70/30 blend.
Vascular support made of a MgZn4 alloy, which includes a woven net made of wires of a MgZn2 alloy on the inside, for dilating a vulnerable plaque in a coronary vessel, wherein the net, which is made of wires having a thickness of 20 μm to 50 μm and a mean distance between the wires of approximately 250 μm, prevents components of the vulnerable plaque from reaching the coronary vessel in the event the plaque ruptures.
Vascular support having high stability, made of a MgZn7.5 alloy for sealing a vulnerable plaque in a coronary vessel, which can be used uncoated or containing a layer made of polyhydroxybutyrate; the vessel support has a metal to artery ratio of 35%.

Further examples of the invention using low Zn alloys
Vascular support made of a MgCa0.5Zn0.2 alloy for dilating a vulnerable plaque in a coronary vessel, of which ≥50% of the volume of the body is degraded residue-free within approximately 180 days and ≥80% of the volume of the body is degraded residue-free within 360 days; the vessel support has a metal to artery ratio of 23% to 30%.
Vascular support made of a MgCa0.75Zn0.5 alloy, which can be used as a flow diverter in general for vascular aneurysms and which has a metal to artery ratio of 19%.
Vascular support made of a MgZn1Ca0.3 alloy for dilating a vulnerable plaque in a coronary vessel, of which ≥90% of the volume of the body is degraded without residue in 360 days.

The advantage of the solution according to the invention is that an implant can be provided for the above-described applications which can dissolve rapidly and completely, without residue or with an extremely small amount of degradation products that remain, within a period of time of less than 360 days, or also within a considerably shorter period of time, and thereby avoids undesirable long-term complications or the need for drug therapies, for example for anti-coagulation.

1. A vascular support implant comprising a magnesium alloy, the alloy including Zn and/or one of the following elements: Ca, Sr, wherein the Zn content is ≤1.5 wt. % and a Ca content is ≥0.25 wt. %.

2. The implant of claim 1, comprising one or more micro-alloying elements.

3. The implant of claim 2, wherein the micro-alloying elements comprise less than 1 wt. %.

4. The implant of claim 2, wherein the micro-alloying elements are selected from one or more of the elements Ag, Fe, Mn and Si.

5. The implant of claim 1, wherein the implant has a fine-grained microstructure, having a grain size of no more than 7.5 μm.

6. The implant of claim 1, wherein the implant comprises a net comprising wires.

7. The implant of claim 6, wherein the wires have a diameter which ranges between 10 μm and 100 μm, and/or wherein the preferred size of the mesh (distance between the wires) ranges between 10 μm and 1 mm.

8. The implant of claim 6, wherein the net is coated with a biodegradable polymer.

9. The implant of claim 8, wherein the polymer is selected from the group consisting of polylactide, polyglycolide, polyanhydride, polyhydroxybutyrate, polycaprolactone, polydioxanone, a poly(trimethylene carbonate)-based polymer, polyphosphazene, polyhydroxyalkonates, polyanhydride, polyacetal, polycarbonate, poly(ether ester); a copolymer of the aforementioned polymers, a mixture of the aforementioned polymers, or a blend of the aforementioned polymers.

10. The implant of claim 8, wherein the polymer comprises a thickness between 50 nm to 25 μm.

11. The implant of claim 3, wherein the micro-alloying elements comprise between 0.01 wt. % to 0.1 wt. %.

12. The implant of claim 11, wherein the micro-alloying elements are selected from one or more of the elements Ag, Fe, Mn and Si.

13. The implant of claim 5, wherein the grain size is <5 μm.

14. The implant of claim 13, wherein the grain size is <2.5 μm.
